(19) United States
(12) Patent Application Publication
(10) Pub. No.: US 2002/0137787 A1
(43) Pub. Date: Sep. 26, 2002

(54) Prevention or treatment of complications related to non-insulin dependent diabetes mellitus or type 2 diabetes mellitus

(76) Inventors: W. Blair Geho, Wooster, OH (US); Hans C. Geho, Solon, OH (US)

Correspondence Address: SDG, INC., HANS C. GEHO, 1350 EUCLID AVENUE, SUITE 200, CLEVELAND, OH, 44115, US

(21) Appl. No.: 09/812,410

(22) Filed: Mar. 21, 2001

(51) Int. Cl. A61K 31/4045

(52) U.S. Cl. 514/415

(57) ABSTRACT

A method of preventing, controlling, or treating the complications of type 2 diabetes mellitus is disclosed. The method comprises administering serotonin or an analogue therof to the liver of a patient.

### BACKGROUND OF THE INVENTION

1. Field of the Invention

This invention relates to a method of preventing, controlling or treating the disorders related to non-insulin dependent diabetes mellitus (the defined term NIDDM has recently been replaced by type 2 diabetes mellitus, and references to type 2 diabetes herein will be assumed to include NIDDM), and, in particular, preventing, controlling, or treating such disease and a patient suffering therefrom by providing serotonin to the liver of the patient.

2. Description of the Related Art

Type 2 diabetes is associated with a variety of metabolic disorders that include glucotoxicity (which may lead to worsened hypoglycemia), disorders of pancreatic insulin secretion, insulin resistance, dyslipidemia, obesity, neuropathy, retinopathy, nephropathy, disorders of fibrinolysis, hypertension, and cardiovascular disease (these various disorders will be referred to as type 2 diabetes “complications”). It is understood that some of these disorders may precede the onset of frank type 2 diabetes. It is further understood that although the incidence of some complications may differ from one type of diabetes to another, their clinical expression is largely identical. An etiology of type 2 diabetes hyperglycemia was discovered in the 1980s by Dr. W. Blair Geho, and his explanation of type 2 diabetes and a method for treatment of a hormonal deficiency of type 2 diabetes are provided in U.S. Pat. No. 4,761,287.

Heretofore, however, the causal relationship between hepatic serotonin deficiency and type 2 diabetes complications has not been explained. To understand this relationship, it is important to present, in summary fashion, what is known about these complications in the context of type 2 diabetes.

The prevelance of type 2 diabetes in the United States is commonly attributed to an aging population that is also increasingly sedentary and obese. Compared to normal-weight individuals, obese people (i.e., those with a body mass index greater than 300) are at 10-20 times greater risk for type 2 diabetes. Almost 90 percent of type 2 diabetes patients are obese. Despite this prevalence there is not a uniformly accepted explanation for the association between obesity and type 2 diabetes.

Accompanying the obese state in type 2 diabetes patients is often a state of dyslipidemia. In the fasting state, when insulin and glucose levels are low, triglycerides stored in the adipocytes are released because of unopposed lipolytic effects of catecholamines, cortisol, growth hormone(s) and glucagons [See pp. 1496-1507 Goodman & Gilman′ The Pharmacological Basis of Therapeutics 9^{th} Edition]. This leads, in the case of the obese type 2 diabetes patient with excessive adipose tissue mass, to elevated circulating VLDL and triglyceride levels, atherogenic LDL particles, and decreased HDL cholesterol [See Scientific American Medicine, 9 Metabolism VI, p. 9, and Walden, C E: Sex differences in the effect of diabetes mellitus on lipoprotein triglyceride and cholesterol concentrations. New England Journal of Medicine 311:953; 1984]. This increased release of free fatty acids from adipose tissue, with direct delivery to the liver and pancreas, is believed to underlie abnormal lipid metabolism and insulin resistance.

It is not known whether hyperglycemia itself causes many of the microvascular and macrovascular complications associated with type 2 diabetes. There is evidence, supported by animal studies and interventional studies relating to the Diabetes Control and Complications Trial (DCCT) study in humans, that intensive control of blood sugar levels can prevent or control the development of these complications, suggesting a central role for hyperglycemia in the etiology of type 2 diabetes complications.

### SUMMARY OF THE INVENTION

This invention relates to a method of preventing, controlling or treating the complications of type 2 diabetes mellitus, and, in particular, preventing, controlling or treating such complications in a patient suffering therefrom by providing serotonin, a serotonin analogue, as defined herein, or a serotonin reuptake inhibitor, to the liver of the patient. Additionally, a method is disclosed of treating type 2 diabetes complications in the presence of hyperglycemia and normal or high levels of insulin available to the liver by such utilization of serotonin, a serotonin analogue, or a serotonin reuptake inhibitor.

### DETAILED DESCRIPTION OF THE INVENTION

The present invention relates to a method of preventing, controlling or treating the complications of type 2 diabetes in a mammal, e.g., a human being or an animal. The method comprises providing serotonin, a serotonin analogue, or a serotonin reuptake inhibitor to the liver of such mammal.

U.S. Pat. No. 4,761,287, which is incorporated hereinto by reference in its entirety, reveals that serotonin is released by the enterochromaffin cells, or the gut enterochromaffin gland, into the portal circulation for ultimate delivery to the liver upon vagal stimulation that precedes and/or accompanies ingestion of a meal; and that it is a necessary cofactor, along with insulin, in normal liver metabolism of glucose.

The failure of serotonin to be released into the liver at mealtime results in the undesirable post-prandial and near post-feeding metabolic state of net hepatic glucose output.

The present invention further provides that the chronic condition of type 2 diabetes caused by a paucity of hepatic serotonin results not only in hyperglycemia but also in the conditions of hyperinsulinemia, insulin resistance, obesity, dyslipidemia, hypertension, disorders in fibrinolysis, atherosclerosis, retinopathy, nephropathy, and cardiovascular disease.

When serotonin is unavailable to the liver in the fed state, insulin alone is not sufficient to cause normal net hepatic glucose retention or uptake. In the feeding patient with adequate levels of insulin but inadequate levels of endogenous hepatic serotonin, the abnormal fed state of net hepatic glucose output occurs. Net hepatic glucose output results in increased blood glucose, or hyperglycemia. These patients are referred to as type 2 (or non-insulin dependent) diabetics. The type 2 diabetic's body responds to this hyperglycemia by releasing increased amounts of insulin, to drive the excess glucose into the body's peripheral muscle and fat (adipose) cells. In the adipose cells, this excess glucose is converted to fat by known biochemical processes. Over time, this method used by the body to metabolize excess blood glucose contributes to obesity, and is a contributing factor to dyslipidemia and hypertension.

Certain medications may cause or contribute to obesity, hyperglycemia, and dyslipidemia. This offers a supportive insight into the etiology of type 2 diabetes complications and the role of hepatic serotonin. For example, the psychotherapeutic agent Clozapine, a serotonin receptor antagonist, has been noted to cause patients to develop hyperglycemia, elevated triglyceride levels, and increased weight. In the central nervous system, the effect of Clozapine is the reversal of depression. Peripherally, however, its net effect is hyperglycemia. Therein lies the genesis of serotonin antagonist-related weight gain: blockade of serotonin's portal or hepatic effect in the fed state prevents normal net hepatic glucose uptake, leading in turn to elevated blood glucose. This then leads to enhanced release of insulin, which helps drive excess blood glucose into adipose tissue, enhancing an increasingly anabolic effect. Over time, the patient gains weight and develops and abnormal lipid profile due to the abnormal release of free fatty acids from adipose tissue to the pancreas and the liver.

Conversely, however, weight gain is not a noted side effect of selective serotonin reuptake inhibitors. In fact, Fluoxetine has been shown to lead to weight loss in overweight depressed patients. Fluoxetine is also reported to have positive effects on blood glucose and lipid profiles as well. The anti-diabetic effect of Fluoxetine and other agents that enhance or potentiate the effect of serotonin is probably due to the enhanced or potentiated effect of serotonin on the liver. When the type 2 diabetic patient taking Fluoxetine eats, their pancreas releases insulin. The Fluoxetine causes increased circulating levels of endogenous serotonin. Together, insulin and serotonin convert the liver to net glucose reuptake. Prevention, treatment or control of type 2 diabetes complications with Fluoxetine, however, is not optimal because of its non-hepatic side effects.

As revealed in medical literature, prolonged hyperglycemia is thought to play a role in the pathogenesis of microvascular and macrovascular disease in type 2 diabetic patients (this theory is sometimes referred to as the “glucose hypothesis”). Some of these diseases are also attributed to insulin resistance or even hyperinsulinemia. Another aspect of the present invention is the prevention, control, or treatment of atherosclerosis, hypertension, retinopathy, nephropathy, hyperinsulinemia, insulin resistance, disordered fibrinolysis, and cardiovascular disease in type 2 diabetic patients by the elimination of prolonged hyperglycemia using the described administration of serotonin or a serotonin analogue.

Over time, a type 2 diabetic patient treated with either a hepatic serotonin replacement or a serotonin analogue, as defined herein, of the present invention is expected to develop a normal pattern of glucose metabolism with improved weight control and an improved lipid profile, and to have a lower relative risk of developing other type 2 diabetes risk factors and complications.

The term “serotonin” shall be interpreted to mean 5-hydroxytryptamine (“5HT”) and it also includes any and all “serotonin analogues” or “serotonergic analogues” which are defined as any and all agents that act as agonists at hepatic 5HT receptors and their subtypes, including but not limited to those 5HT receptors identified as 5-HT1A, 5-HT1B, 5-HT1C, 5-HT1D, and all other 5-HT1 receptor subtypes, 5-HT2A, 5-HT2B, 5-HT2C, and all other 5-HT2 receptor subtypes, 5-HT3 and all 5-HT3 receptor subtypes, 5-HT4 and all 5-HT4 receptor subtypes, 5-HT5 and all 5-HT5 receptor subtypes, 5-HT6 and all 5-HT6 receptor subtypes, and 5-HT7 and all 5-HT7 receptor subtypes.

In addition, for purposes herein, “serotonin” shall also be interpreted to mean “serotonin pro-drugs” which are defined as serotonin covalently or ionically-linked to a suitable in vivo pharmaceutical carrier, which carrier releases the serotonin at a receptor site in the liver.

Further, for the purposes herein, “serotonin analogues” will also be defined as any agent that either acts as an agonist at serotonin receptors (i.e., “serotonergic agent”) or potentiates the activity of serotonin or serotonergic agents at hepatic serotonin receptors, including but not limited to agents of the following classes:

Class 1: Serotonin precursors including but not limited to tryptophan.

Class 2: Serotonin analogues that are of a class whose activity are blocked by serotonin receptor antagonists such as but not limited to methysergide, ketanserin, clozapine, resperidone, and cryoheptadine.

Class 3: Serotonin receptor agonists such as but limited to buspirone, ergot alkaloids, sumatriptan, cisapride, D-Lysergic Acid Diethylainide (“LSD”), 8-Hydroxy-(2-N,N-Dipropylamino)-Tetraline (8-OH-DPAT), and m-Chlorophenylpiperazine (“mCPP”).

Class 4: Agents that block the reuptake of serotonin, including but not limited monoamine oxidase inhibitors (“MAOI”) such as Phenelzine, tranylcypromine, selegiline, and isocarboxacid; and tricyclic antidepressants such as amitryptiline, amoxapine, clomipramine, desipramine, doxepin, imipramine, maprotiline, nortriptyline, protriptyline, and trimipramine.

Class 5: Agents that selectively inhibit serotonin reuptake, also known as Selective Reuptake Inhibitors, or “SSRIs” including, but not limited to fluoxetine, sertraline, paroxetine, and fluvoxamine.

Class 6: Agents that enhance serotonin release, including but not limited to d-amphetamine, reserpine, methylphenidate, and pemoline.

Class 7: Over the years, there have been a number of agents discovered that are clearly classed as serotonin receptor agonists, serotonin reuptake inhibitors, SSRIs, MAOIs, or serotonin release enhancers; however, there are other new agents that do not fall clearly into these identified classes. These agents are defined as atypical or second generation agents, and include but are not limited to buproprion, nefazadone, trazadone, and venlafaxine. In addition, some metabolites of these agents, such as m-chlorophenylpiperazine, a metabolite of trazadone, act as serotonin agonists.

For convenience purposes herein only, whenever the term “serotonin” is employed, it shall encompass any of the agents or components of a “serotonin” or “serotonin analogue” as it is defined above.

An effective amount of serotonin is selected. The serotonin is administered to a patient in need for preventing, controlling or treating the complications of type 2 diabetes. The serotonin is delivered to the liver preferably by means of a Hepatic Directed Delivery System (“HDD”), which may be, but is not limited to, a liposome, a polymer, or a combined form of a lipid-polymeric vehicle.

The serotonin is given to a patient to be treated at or around mealtime, or as a basal dose, using conventional means such as oral, subcutaneous, intravenous, topical, inhaled, or suppository administration. To insure directed administration to the liver, a preferred method is to deliver via any of these routes using a hepatic-targeted delivery system such as the HDD.

The preparation of certain HDD systems is well known. In this regard, reference is made to U.S. Pat. Nos. 4,337,567; 4,603,044; 5,104,661; and 6,063,400 as well as pending U.S. patent applications Ser. Nos. 09/313,828, 09/109,473 and 09/122,272, all of which are incorporated hereinto by reference in their entirety.

The effective amount of hepatically-directed serotonin or serotonin analogue that is administered will of course be dependent on the subject being treated, the type and severity of the affliction, the manner of administration and the judgment of the prescribing physician. Although the effective dosage ranges are dependent upon a variety of factors, and are generally known to one of ordinary skill in the art, some dosage guidelines can be generally defined.

Typically, the hepatically-directed serotonin or serotonin analogue is present in the bottle, syringe, capsule or other pre-administration container in an amount of 0.01 to 200 mg/mL. Preferably, the effective amount to be delivered to the patient for the prevention, control, or treatment of type 2 diabetes complications is from about 0.01 micrograms to about 50 miligrams per kilogram of body weight of the patient of the serotonin or the serotonin analogue.

For best results in preventing, controlling or treating the complications of type 2 diabetes, the patient in need thereof should have the serotonin or the serotonin analogue administered to him or her prior to or simultaneously with the administration of food.

1. A method of preventing, controlling, or treating the complications of type 2 diabetes mellitus, including hypoglycemia, hyperinsulinemia, insulin resistance, microvascular and macrovascular disease, visual impairment, blindness, nephropathy, renal failure, hypertension, stroke, atherosclerosis, cardiovascular disease, neuropathy, abnormal hemostasis and immune functions, which comprises administering serotonin or a serotonin analogue to the liver.

2. A method as defined in claim 1 wherein the serotonin or serotonin analogue is delivered to the liver by means of a HDD or other suitable pharmaceutical carrier.

3. A method as defined in claim 1 wherein the serotonin or serotonin analogue is delivered to the liver by means of a serotonin precursor such as a serotonin prodrug, a serotonin reuptake inhibitor, a serotonin release enhancer, a serotonin receptor antagonist, a monoamine oxidase inhibitor, a tricyclic antidepressant, or an atypical or second generation antidepressant agent.

4. A method as defined in claim 1 wherein the hepatic receptor for serotonin or serotonin analogues is activated or potentiated.
